(19) United States
(12) Patent Application Publication
(10) Pub. No.: US 2008/0015597 A1
(43) Pub. Date: Jan. 17, 2008

(54) LARGE DIAMETER BONE ANCHOR ASSEMBLY

(76) Inventor: Dale E. Whipple, East Taunton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/741,135

(22) Filed: Apr. 27, 2007

Related U.S. Application Data
(60) Provisional application No. 60/796,044, filed on Apr. 28, 2006.

(51) Int. Cl. A61B 17/58

(52) U.S. Cl. 606/73; 606/72

(57) ABSTRACT

A bone anchor assembly is described having a large diameter for fixing a spinal connection element to bone. The assembly includes a receiver member for receiving the spinal connection element, a bone-engaging shank for engaging bone, a plurality of inserts for retaining the head of the shank within the receiver member and a retaining piece for retaining the inserts within the receiver member.

### CONTINUING DATA

This application claims the benefit of U.S. Provisional Patent Application Ser. No. 60/796,044, entitled “Large Diameter Bone Anchor Assembly”, filed Apr. 28 2006, which is hereby incorporated herein by reference.

### BACKGROUND

Spinal connection systems may be used in orthopedic surgery to align and/or fix a desired relationship between adjacent vertebrae. Such systems typically include a spinal connection element, such as a relatively rigid fixation rod or plate or a dynamic connector, that is coupled to adjacent vertebrae by attaching the element to various anchoring devices, such as hooks, bolts, wires, or screws. The spinal connection element can have a predetermined contour that has been designed according to the properties of the target implantation site, and once installed, the spinal connection element holds the vertebrae in a desired spatial relationship, either until desired healing or spinal fusion has taken place, or for some longer period of time.

Spinal connection elements can be anchored to specific portions of the vertebra. Since each vertebra varies in shape and size, a variety of anchoring devices have been developed to facilitate engagement of a particular portion of the bone. Pedicle screw assemblies, for example, have a shape and size that is configured to engage pedicle bone. Such screws typically include a threaded shank that is adapted to be threaded into a vertebra, and a head portion having a spinal connection element receiving portion, which, in spinal rod applications, is usually in the form of a U-shaped slot formed in the head for receiving the rod. A set-screw, plug, cap or similar type of closure mechanism, may be used to lock the connection element into the connection element receiving portion of the pedicle screw. In use, the shank portion of each screw may be threaded into a vertebra, and once properly positioned, a connection element may be seated through the spinal connection element receiving portion of each screw and the connection element is locked in place by tightening a cap or similar type of closure mechanism to securely interconnect each screw and the connection element. Other anchoring devices also include hooks and other types of bone screws.

In certain procedures, such as those in the lumbar or sacral spine, it may be necessary to use a larger diameter pedicle screw capable of carrying large loads or engaging large pedicles. A difficulty in using a larger diameter screw comes from the corresponding increase in the size of the receiver head to accommodate the larger diameter screw shank, since the shank is usually assembled from the top through the opening at the proximal end of the receiver head. The increased size of the receiver head can interfere with the bony anatomy and can limit the polyaxial range of motion of the screw head. Another problem associated with manufacturing large diameter top-loading screws is that the opening in the receiver head has to be larger to accept the larger diameter screw shank, which creates the need for a larger closure mechanism. It is desirable to maintain the same size opening in the receiver head such that the same size closure mechanisms can be used. Accordingly, a larger diameter polyaxial screw is needed which is not top-loading.

### SUMMARY

Disclosed herein are embodiments of a bottom-loading bone anchor assembly having a large diameter bone-engaging shank. In one embodiment, the bone anchor assembly for engagement to a connection element includes a receiver member having an opening at the proximal end for receiving the connection element and a bore having a diameter greater than the opening; a bone-engaging shank having a head at a proximal end, the head sized to fit through the bore of the receiver member; and a plurality of inserts having a shape to accommodate the head of the shank, and sized to fit within the receiver member to retain the head of the shank within the receiver member; and a retaining piece sized to fit proximal to the inserts and engaging the receiver member to retain the inserts within the receiver member.

### BRIEF DESCRIPTION OF THE FIGURES

These and other features and advantages of the bone anchor assembly and methods disclosed herein will be more fully understood by reference to the following detailed description in conjunction with the attached drawings in which like reference numerals refer to like elements through the different views. The drawings illustrate principles of the bone anchor assembly and methods disclosed herein and, although not to scale, show relative dimensions.

FIG. 1A illustrates an exploded view of a large diameter bone anchor assembly.

FIG. 1B illustrates a cross-section of the bone anchor assembly shown in FIG. 1A.

FIG. 1C illustrates a top view of the bone anchor assembly shown in FIG. 1A.

FIG. 1D illustrates a perspective view of the insert being positioned between the receiver member and the bone-engaging shank of the assembly in FIG. 1A.

FIG. 1E illustrates an exploded view of the inserts prior to assembly.

FIG. 1F illustrates an assembled view of the inserts with the retaining piece from FIG. 1A.

FIG. 1G illustrates insertion of the insert within the receiver member of FIG. 1A.

FIG. 1H illustrates a cut away view of the receiver member with the inserts assembled and prior to insertion of the retaining piece shown in FIG. 1A.

FIG. 2A illustrates a perspective view of the insert of the bone anchor assembly shown in FIG. 1A.

FIG. 2B illustrates a perspective view of a second insert of the bone anchor assembly shown in FIG. 1A.

FIG. 2C illustrates a cross-section view of the insert shown in FIG. 2A.

FIG. 3A illustrates an isometric view of the receiver member of the bone anchor assembly shown in FIG. 1A.

FIG. 3B illustrates a cross-section view of the receiver member of the bone anchor assembly shown in FIG. 1A.

FIG. 3C illustrates a cross-section view of the receiver member taken along the leg.

FIG. 4 illustrates a perspective view of the retaining piece of the bone anchor assembly shown in FIG. 1A.

FIG. 5A illustrates a perspective view of the compression member of the bone anchor assembly shown in FIG. 1A.

FIG. 5B illustrates a cross-section view of the compression member of the bone anchor assembly shown in FIG. 1A.

FIG. 5C illustrates a top view of the compression member of the bone anchor assembly shown in FIG. 1A.

### DETAIL DESCRIPTION OF EXEMPLARY EMBODIMENTS

Certain exemplary embodiments will now be described to provide an overall understanding of the principles of the structure, function, manufacture, and use of the large diameter bone anchor assembly and methods disclosed herein. One or more examples of these embodiments are illustrated in the accompanying drawings. Those of ordinary skill in the art will understand that the large diameter bone anchor assembly and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

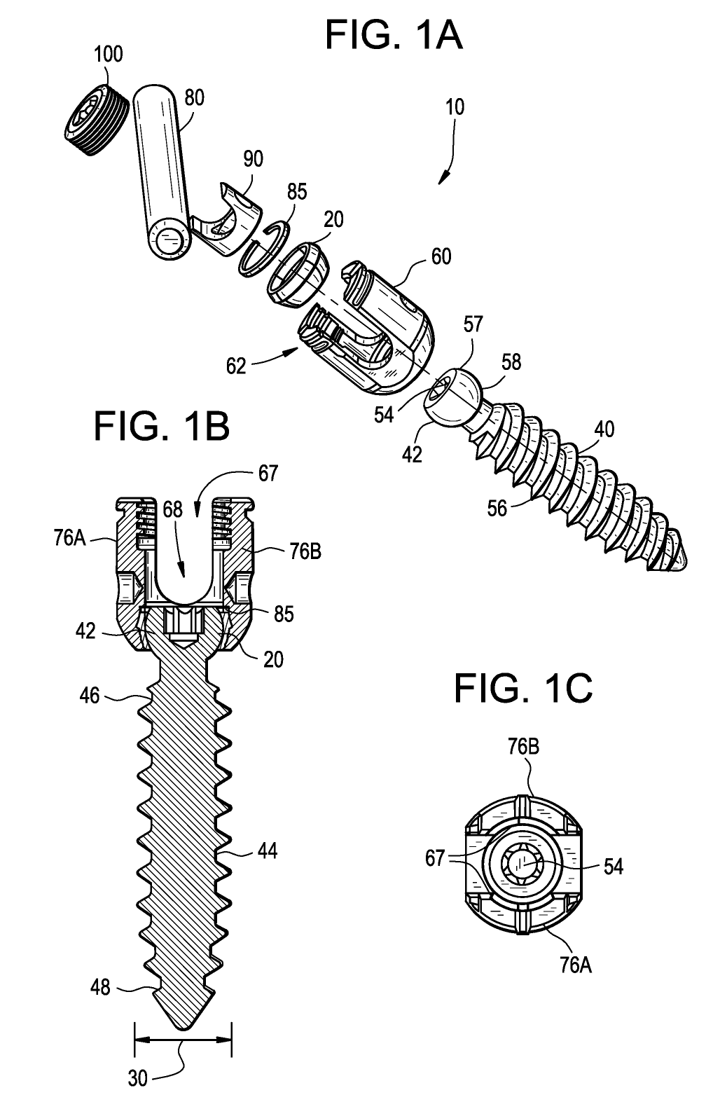

FIGS. 1-5 illustrate an exemplary embodiment of a bottom-loading large diameter bone anchor assembly. The exemplary bone anchor assembly 10 may be employed to engage one or more spinal connection elements to bone. For example, bone anchor assembly 10 may be employed to connect a spinal plate, rod (rigid or dynamic), and/or cable to a vertebra of the spine. Although the exemplary bone anchor assembly 10 described below is designed primarily for use in spinal applications, one skilled in the art will appreciate that the structure, features, and principles of the exemplary bone anchor assembly 10, as well as the other exemplary embodiments described below, may be employed to couple any type of orthopedic implant to any type of bone or tissue. Non-limiting examples of applications of the bone connection anchor assembly 10 described herein include long bone fracture fixation/stabilization, small bone stabilization, lumbar spine as well as thoracic stabilization/fusion, cervical spine compression/fixation, dynamic, non-fusion applications including facet replacement and dynamic posterior systems as well as skull fracture/reconstruction plating.

The illustrated exemplary bone anchor assembly 10 includes a bone-engaging shank 40 configured for engaging bone, a receiver member 60 for receiving a spinal connection element, and an insert 20 for retaining the shank 40 within the receiver member 60. The bone-engaging shank 40 extends from a proximal end 46 to a distal end 48 along a longitudinal axis. An outer surface 44 of the bone-engaging shank 40 extends between the proximal end 46 and the distal end 48. The outer surface 44 of the bone-engaging shank 40 may include one or more bone engagement mechanisms to facilitate gripping engagement of the bone anchor assembly 10 to bone. In the illustrated exemplary embodiment, for example, the bone-engaging shank 40 includes an external thread 56. The external thread 56 may extend along at least a portion of the bone-engaging shank 40. For example, in the illustrated exemplary embodiment, the external thread 56 extends from the distal end 48 to the proximal end 46 of the bone-engaging shank 40. One skilled in the art will appreciate that bone engagement mechanisms other than external thread 56 may be employed, including, for example, one or more annular ridges, multiple threads, dual lead threads, variable pitched threads, and/or any other conventional bone engagement mechanism. In the illustrated exemplary embodiment, the shank diameter 30 of bone-engaging shank 40 may be defined by the major diameter of external thread 56.

The proximal end 46 of the exemplary bone-engaging shank 40 has a head 42 configured to fit within the receiver member 60 and to facilitate adjustment of the shank 40 relative to the receiver member 60. For example, the head 42 may be generally spherical in shape to permit pivoting of the bone-engaging shank 40 relative to the receiver member 60. In the illustrated exemplary embodiment, for example, the head 42 may be in the shape of a truncated sphere having a generally planar proximal surface 57 and a generally hemispherically shaped distal surface 58. The head 42 of the shank 40 may have surface texturing, knurling, and/or ridges. A drive feature 54 may be located internally or externally on the head 42 of the shank 40.

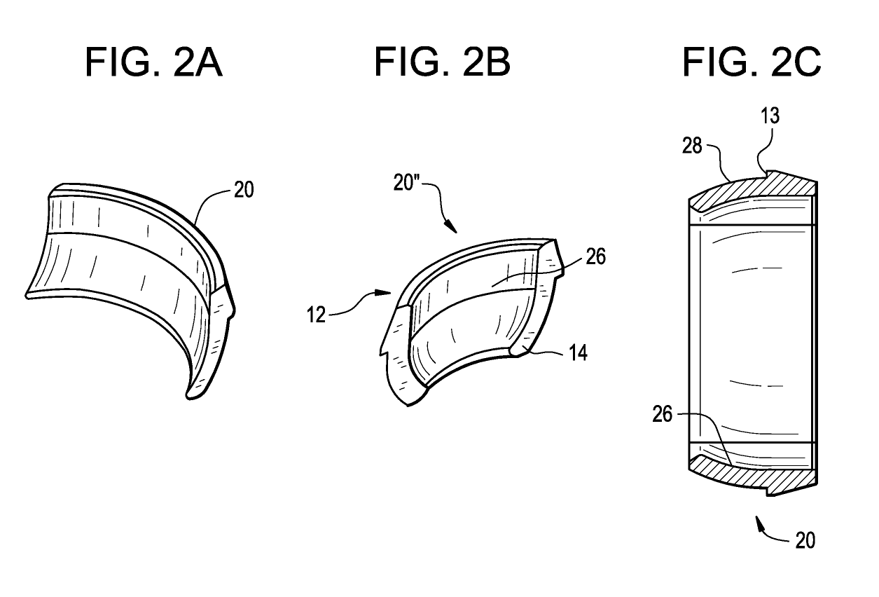

Referring to FIGS. 3A-C, the receiver member 60 of the exemplary bone anchor assembly 10 includes a proximal end 62 having a cylindrical opening 67 leading to recess 68, and a distal end 70 having a bore 64 forming a seat portion 72. The receiver member 60, in certain exemplary embodiments, may be configured to receive a spinal connection element and couple the spinal connection element to the bone anchor assembly. In the exemplary embodiment, for example, the recess 68 of the receiver member 60 may be sized and shaped to receive a spinal rod 80, as illustrated in FIG. 1A. For example, the receiver member 60 has a generally U-shaped cross-section defined by two legs 76A and 76B separated by recess 68. Each leg 76A, 76B is free at the proximal end 62 of the receiver member 60. In the exemplary embodiment, for example, the inner surfaces of the legs 76A, 76B are threaded to mate with a corresponding thread on the closure mechanism shown as a setscrew. The exemplary spinal rod 80 may be seated within the recess 68 by aligning the spinal rod 80 and the recess 68, and advancing the spinal rod 80 between the legs 76A, 76B into the recess 68. The configuration of recess 68 of the receiver member 60 may be varied to accommodate the type, size and shape of spinal connection element employed.

In the exemplary embodiment, the bore 64 of the receiver member 60 forms a seat portion 72 within the receiver member 60. The bore 64 is sized to allow at least a portion of a bone anchor assembly, such as the head 42 of the shank 40 to pass through to the seat portion 72. For example, the head 42 of the shank 40 may be inserted in the proximal direction through the bore 64 of the receiver member 60, as illustrated in FIG. 1A. The seat portion 72 has a diameter greater than the bore 64 and the cylindrical opening 67.

In some exemplary embodiments, the seat portion 72 may be generally spherical in shape to permit pivoting of the bone-engaging shank 40 relative to the receiver member 60. In the illustrated exemplary embodiment, the seat portion 72 may have a proximal portion 73 and a distal portion 71. The distal portion 71 may be generally spherical in shape and may have a curvature analogous to the distal surface 58 of the head 42 of the shank 40. The proximal portion 73 may have a diameter greater than the distal portion 71 and taper towards the proximal end of the receiver member 60. The proximal portion 73 of the seat portion 72 may have a diameter greater than the bore 64. Adjacent the proximal seat portion 73 of the receiver member 60 is another recess 75 adapted to accommodate the locking ring 85 described in more detail below. The recess 75 may have a circular configuration. In other exemplary embodiments, the seat portion 72 may be tapered or may have any other shape that allows adjustment of the head 42 of the shank 40 relative to the receiver member 60. In the exemplary embodiment, the bone anchor assembly 10 is a polyaxial bone anchor assembly. The bone-engaging shank 40 when assembled within the receiver member 60 may be pivoted to one or more angles relative to the receiver member 60.

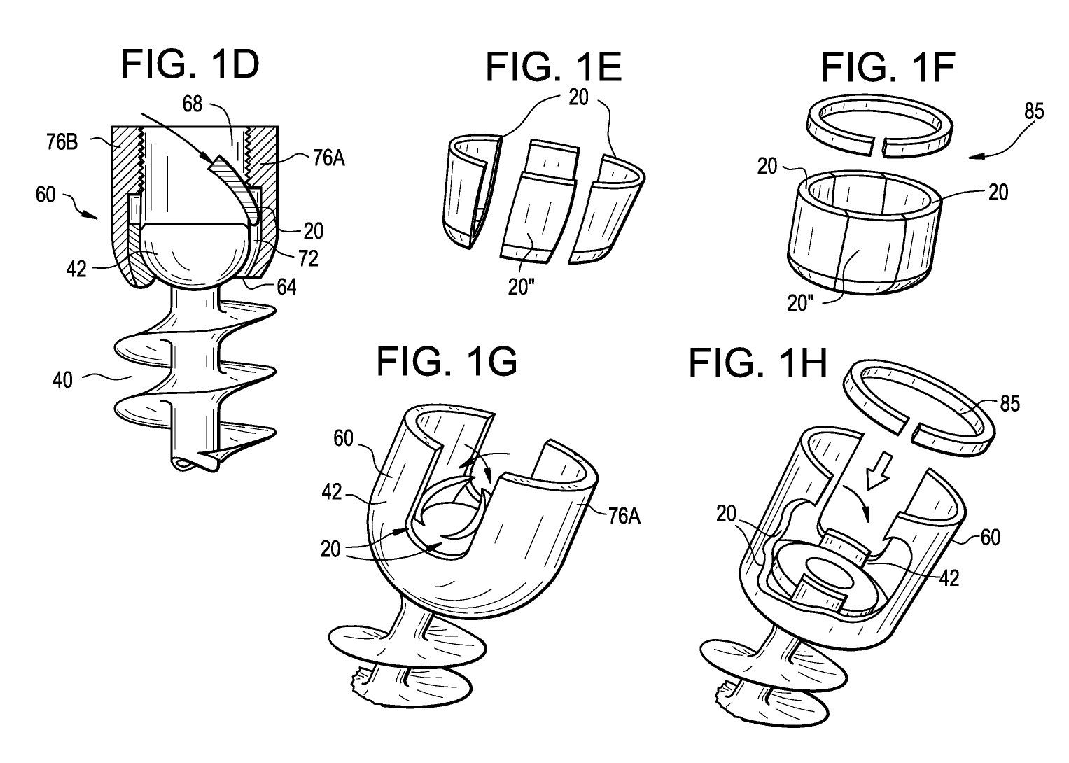

Referring to FIG. 1B, first insert 20 of the bone anchor assembly 10 is positionable within the seat portion 72 of the receiver member 60. The insert 20 may have an inner surface 26 contoured for engaging the head 42 of the shank 40 and an outer surface 28 for engaging the seat portion 72 of the receiver member 60. The inner surface 26 may have a semi-spherical shape in the exemplary embodiment, while the outer surface 28 has a generally semi-circular shape as illustrated in FIG. 2C. The inner surface 26 is shaped to advance around the head 42 of the shank 40 during insertion within the seat portion 72 of the receiver member 60. The insert 20 extends from a proximal end 12 to a distal end 14. The outer surface 28 at the proximal end 12 of the insert 20 may project further than the distal end 14 creating a lip 13. The lip 13 may be sized to fit within the proximal portion 73 of the seat portion 72 of the receiver member 60. The insert 20 may taper from the proximal end 12 of the insert 20 to the distal end of the insert 20. In one exemplary embodiment there may be more than one insert, the second insert 20″ may have the same shape but have a different size than the first insert 20 as seen in FIGS. 2A-B. The inserts 20, 20″ retain the head 42 of the shank 40 within the receiver member 60. In the illustrated exemplary embodiment shown in FIG. IE and IF, a pair of first inserts 20 having a first size are positioned opposite each other with a pair of second inserts 20″ around the head 42 of the shank 40 to create a socket within the seat portion 72 of the receiver member 60 for retaining the head 42 of the shank 40. One skilled in the art will recognize that any number of inserts could be used to assemble a socket to retain the head within the receiver member.

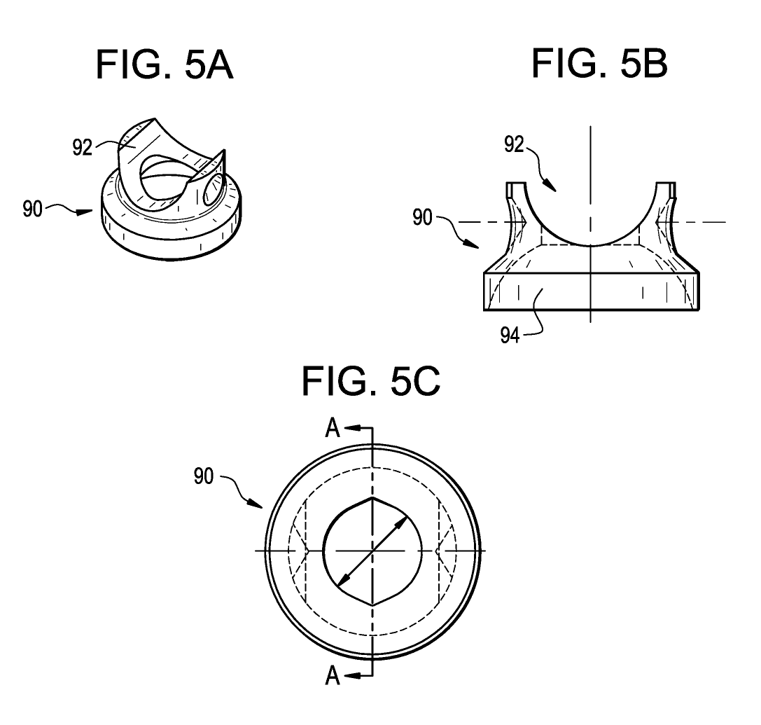

The bone anchor assembly 10 may include a compression member 90 as shown in FIGS. 5A-C positionable within the receiver member 60 between the spinal connection element and the bone anchor. As illustrated in FIG. 1B, the compression member 90 may be positioned within the recess 68 between the spinal rod 80 and the head 42 of the shank 40. In the exemplary embodiment, the compression member 90 may have a proximal first surface 92 for engaging the spinal connection element and an opposing distal second surface 94 for engaging the head 42 of the shank.

The exemplary bone anchor assembly 10 may include a closure mechanism 100 that secures the spinal connection element to the bone anchor assembly. Referring to FIG. 1A, the closure mechanism 100 secures the exemplary spinal rod 80 within the recess 68 of the receiver member 60. The closure mechanism 100 may engage the proximal end 62 of the receiver member 60 or, in other exemplary embodiments, may engage other portion(s) of the receiver member 60. The exemplary closure mechanism 100 is an internal setscrew that engages an inner surface of the first end 62 of the receiver member 60. For example, the closure mechanism 100 may have external threads 102 that engage internal threads 104 provided at the proximal end 62 of the receiving member 60. Distal advancement of the closure mechanism 100 into engagement of the spinal rod 80, seats the spinal rod 80 in the proximal surface 92 of the compression member 90. The compression member 90 is advanced over a portion of the head 42 of the bone-engaging shank 40 pushing the head 42 distally within the inserts 20 and thereby fixing the relative movement of the head 42 in relation to the receiver member 60. In one embodiment, the major diameter of the bone-engaging shank 30 may be greater than the diameter of the closure mechanism 100 and the cylindrical opening 67 at the proximal end 62 of the receiver member 60.

One skilled in the art will appreciate that other types of closure mechanisms may be employed. For example, an external closure mechanism positionable around the outer surface of the legs 76A, 76B of the receiving member 60 may be employed. In other exemplary embodiments, the closure mechanism may comprise an external and an internal closure mechanism, a non-threaded twist-in cap, and/or any other conventional closure mechanism.

The components of the bone anchor assembly may be manufactured from any biocompatible material, including, for example, metals and metal alloys such as titanium and stainless steel, polymers, and/or ceramics. The components may be manufactured of the same or different materials. In one exemplary method of manufacturing, the bone-engaging shank 40, the insert 20 and the receiver member 60 are separately constructed and assembled prior to implantation. The head 42 of the shank 40 is inserted proximally through the bore 64 of the receiver member 60. The first insert 20 in one exemplary method may be inserted within the receiver member 60 through the cylindrical opening 67 at the proximal end 62 extending into the recess 68. The distal end of the insert 20 advances around the head 42 until it is seated within the seat portion 72 of the receiver member 60. If more than one insert is used, a second first insert 20 is then inserted in the same manner. In one exemplary embodiment, a second pair of inserts 20″ may also be inserted through the proximal end 62 of the receiver member 60 around the head 42 of the shank 40 until the insert 20″ is fully seated within the seat portion 72. The compression member 90 is swaged into position between the legs 76A, 76B of the receiver member 60.

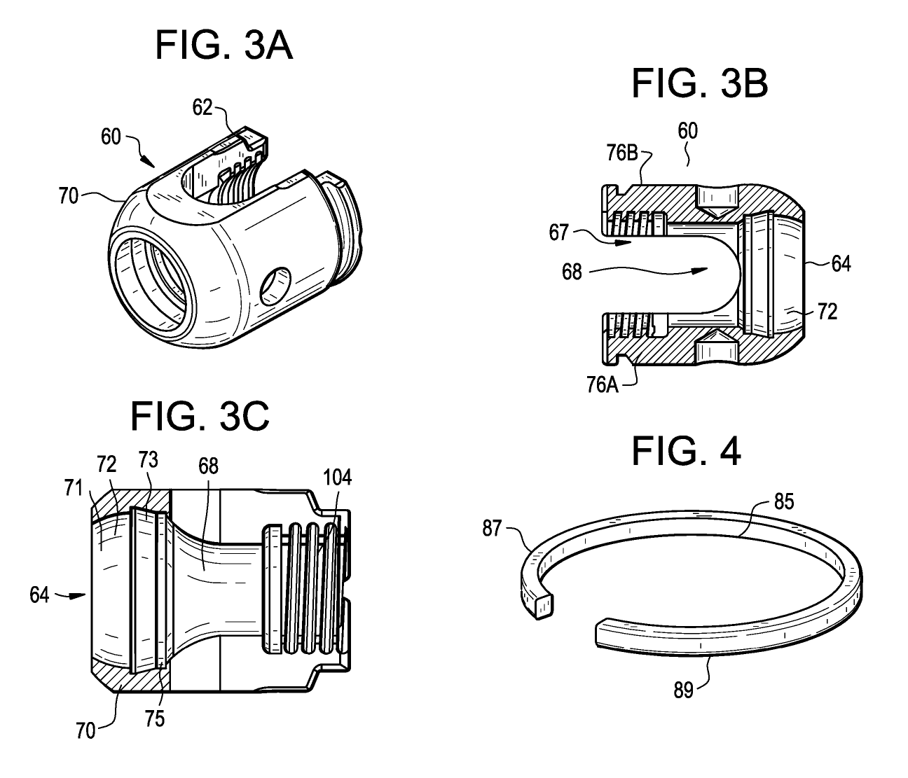

Referring to FIG. 4, a retaining piece 85 may be used to keep the inserts 20, 20″ contained within the seat portion 72 of the receiver member 60. The retaining piece 85 may have a generally C-shape having a first diameter. The retaining piece 85 may be compressed to a second smaller diameter to fit through the opening at the proximal end 62 of the receiver member 60 and advanced into position within the recess 75 adjacent the seat portion 72 of the receiver member 60. The retaining piece 85 expands back to the first diameter to fit within the recess 75. The distal end 89 of the retaining piece 85 contacts the proximal surface of the inserts 20, 20″ and the proximal end 87 of the retaining piece 85 engages the proximal end of the recess 75 within the receiver member, preventing the inserts 20, 20″from advancing proximally into the recess 68 of the receiver member 60. The retaining piece 85 may be constructed from an elastomeric material, such as rubber, or may be constructed from a metal such as stainless steel and titanium, or other suitable material. In alternative embodiments, the retaining piece 85 may be positioned within a groove provided in the outer surface 28 of the insert(s) (20). In such embodiments, the retaining piece 85 may also seat within recess 75 or, alternatively, recess 75 may be eliminated and retaining piece 85 may seat within the groove in the insert(s) 20 and contact the inner wall of the receiver member 60.

While the large diameter bone anchor assembly and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

What is claimed:

1. A bone anchor assembly for engagement to a connection element comprising:
a receiver member having an opening at the proximal end for receiving the connection element and a bore having a diameter greater than the opening;
a bone-engaging shank having a head at a proximal end, the head sized to fit through the bore of the receiver member;
a plurality of inserts having a shape to accommodate the head of the shank, and sized to fit within the receiver member to retain the head of the shank within the receiver member; and
a retaining piece sized to fit proximal to the inserts and engaging the receiver member to retain the inserts within the receiver member.

2. The bone anchor assembly of claim 1, wherein the head of the shank has a generally spherical shape.

3. The bone anchor assembly of claim 2, wherein the insert has an inner surface having a generally semi-spherical shape to accommodate the head of the shank.

4. The bone anchor assembly of claim 1, wherein the insert has a lip on the outer surface for engaging the receiver member.

5. The bone anchor assembly of claim 1, wherein the insert comprises a second pair of inserts having a second size different than the first size.

6. The bone anchor assembly of claim 1, wherein the retaining piece has a distal portion for engaging a proximal portion of the insert to prevent the insert from moving in a proximal direction.

7. The bone anchor assembly of claim 1, further comprising a closure mechanism, wherein the diameter of the head of the shank is greater than the closure mechanism.

8. The bone anchor assembly of claim 7, wherein the major diameter of the shank is greater than the closure mechanism.

9. The bone anchor assembly of claim 1, wherein the major diameter of the shank is greater than the diameter of the opening of the receiver member.
